(19) United States
(12) Patent Application Publication
(10) Pub. No.: US 2025/0099960 A1
(43) Pub. Date: Mar. 27, 2025

(54) Device for Receiving Samples

(71) Applicant: PROCOMCURE BIOTECH GMBH, Thalgau (AT)

(72) Inventor: Kamil ÖNDER, Eugendorf (AT)

(21) Appl. No.: 18/728,716

(22) PCT Filed: Feb. 3, 2022
(86) PCT No.: PCT/EP2022/052588

§ 371 Date: Jul. 12, 2024

(30) Foreign Application Priority Data
Jan. 21, 2022 (EP) 22152660.1

(51) Int. Cl. B01L 3/00; C12Q 1/686; C12Q 1/70

(52) U.S. Cl. CPC B01L 3/5029; B01L 3/50825; C12Q 1/701; B01L 2300/021; B01L 2300/046; B01L 2300/069; C12Q 1/686

(57) ABSTRACT

Device (1) for receiving biological samples for carrying out a PCR analysis method for determination of pathogenic_microorganisms, in particular pathogenic viruses, especially viruses that cause respiratory diseases, having—a receiving container (2) with a first opening (4), and a closure device (6) which is adapted to the opening (4) of the receiving container (2) such that the opening (4) of the receiving container (2) is able to be closed with the closure device (6), wherein the closure device (6) has a through-opening (20), characterized in that the receiving container contains a liquid preparation composition for point-of-care implementation of a PCR test.

The invention relates to a device for receiving samples for performing a PCR analytical method for determining pathogenic microorganisms, in particular, pathogenic viruses, especially viruses causing respiratory diseases. The devices according to the invention are suitable for performing in-situ PCR methods (point of care; PoC PCR). In addition, the invention relates to the use of the device for a PoC PCR test method, and to methods for performing an in-situ PCR method (PoC PCR).

Pathogenic viruses that cause respiratory diseases often spread quickly and may lead to an unclear situation of infection occurrences, up to pandemic conditions. Recently, the spreading of coronaviruses, especially SARS CoV-2 viruses, led to a pandemic with great damage to health and economy. An essential component of controlling the pandemic is a reliable and quick diagnostic method relating to the presence of pathogenic viruses, in order to be able to selectively initiate therapies and measures, and thus control the spreading.

PCR methods, which are usually and on a standard basis performed in specialized laboratories, are considered the reliable gold standard for the detection of pathogenic viruses, especially SARS CoV-2 viruses. However, it is disadvantageous that the performance of such PCR tests is time-consuming, can be performed only by trained personnel, and requires purification as well as transport of the collected patient's samples from the place of sample collection to the laboratory.

Thus, there is a great need for providing reliable and quickly performed tests with respect to the determination of pathogenic viruses.

Surprisingly, it has been found that in-situ (PoC; point of care) PCR tests can be performed when suitable devices for receiving samples are used. In particular, there is an advantage in that the complicated transport of the collected patient's samples is omitted and, in addition, the complicated purification of the samples can be dispensed with. Thus, the PCR tests can be performed close to the patients and directly in situ, for example, in test centers, pharmacies, or doctor's offices.

Thus, the sample collecting devices must be not only practical and simple to use for the in-situ application, but also equipped and designed in such a way that PCR tests can be performed directly in situ.

This object is achieved by the subject matter according to the independent claims of the present invention. The dependent claims reflect advantageous and preferred embodiments of the present invention.

The invention relates to a device for receiving biological samples for performing a PCR analytical method for determining pathogenic microorganisms, in particular, pathogenic viruses, especially viruses causing respiratory diseases, comprising a receiving container with a first opening, a closing means matched to the opening of the receiving container, so that the opening of the receiving container is closable with the closing means, wherein said closing means has a passage opening, and wherein said receiving container contains a liquid processing composition for the in-situ (point of care; PoC) performance of a PCR test.

In an advantageous embodiment, the device further has a sample collecting means that has on a first free end thereof at least one first collecting element, by means of which the sample can be collected, and wherein said sample collecting means can be introduced into the receiving container. This embodiment has the advantage that the device and sample collecting means are together in one system, thus also reducing the risk of contamination in practical application.

Preferably, the sample collecting means is connected with the closing means on a second end that is opposite to a first free end, so that the sample collecting means can be introduced into the receiving container by means of the closing means.

In another preferred embodiment, the passage opening of the closing means is closed by means of a terminating element, wherein said terminating element is detachably attached to said closing element.

In another embodiment, said sample collecting means has at least one hollow rod-shaped element, at the free end of which said collecting element is provided, wherein said hollow rod-shaped element forms a canal inside.

The rod-shaped element can be connected with the closing means in such a way that the canal of the rod-shaped element leads to, or is provided in, the passage opening.

In a preferred embodiment, the passage opening of the closing element is closed on the side opposite of the sample collecting element by means of a terminating element that is detachably attached to the closing element.

For practical reasons of handling, it is advantageous for the terminating element to be separable from the closing element by kinking. In principle, however, further alternatives familiar to those skilled in the art are also possible and usable. Thus, for example, the terminating element may also be detachable by screwing.

In one embodiment, said hollow rod-shaped element has at least one opening at the free end. It is particularly preferred that the end of the canal provided within said rod-shaped element forms the opening at the free end. It is further preferred that the opening provided at the free end is provided in the wall of said rod-shaped element.

Advantageously, the device, especially the receiving container, has a identification code. This enables information relating to the patient to be quickly and safely assigned to the sample to be analyzed. Especially if PCR tests of several samples are performed simultaneously, a confusion in the performance, evaluation and identification can be avoided thereby.

The device of the present invention is suitable, in particular, for the in-situ testing for coronaviruses. Preferably, said viruses are coronaviruses, especially SARS CoV-2 viruses.

In principle, the devices according to the invention can be employed specifically for viruses that cause respiratory diseases. Preferred are one or more viruses selected from the group consisting of SARS CoV-2 virus, influenza virus, and adenovirus.

PCR tests in the field of analyzing and detecting viruses are known to those skilled in the art. Within the scope of the present invention, it is advantageous if said PCR test is a quantitative PCR (qPCR) method, or a quantitative real-time PCR (RT-qPCR).

The biological samples, especially those for examining viruses that cause respiratory diseases, are preferably saliva and/or nasopharyngeal secretion.

An essential component of the present invention is the fact that said receiving container contains a liquid processing composition for the in-situ (point of care; PoC) performance of a PCR test. The processing composition provides that the biological samples can be tested directly in situ by a PCR method without requiring further purification of the sample. Preferably, said liquid processing composition includes guanidinium thiocyanate. The processing composition is preferably an aqueous composition. It has proven advantageous to keep the concentration of guanidinium thiocyanate below 1 M. This improves the direct use of the biological sample together with the processing composition in a PCR test.

In addition, the liquid processing composition preferably includes one or more surfactants.

In addition, the liquid processing composition advantageously includes one or more RNase inhibitors, preferably two different RNase inhibitors.

It has been found advantageous that the liquid processing composition includes a buffer. In particular, this is due to the fact that said biological samples, especially saliva and nasopharyngeal secretion, may have different pH values depending on the patient.

The liquid processing composition preferably includes biotin.

The invention further relates to The use of the device according to the invention for a PoC PCR test method.

The invention further relates to a process for performing an in-situ (PoC PCR) method, comprising the following steps:
a) providing a device according to the invention,
b) contacting a biological sample, preferably saliva and/or nasopharyngeal secretion, with the liquid processing composition for an in-situ (point of care; PoC) performance of a PCR test,
c) using said liquid processing composition including the biological sample, in a PoC PCR method.

In a preferred embodiment, said liquid processing composition is incubated together with the biological sample, preferably incubated at 15 to 30° C., and especially incubated over a period of from 1 to 30 minutes, especially from 2 to 15 minutes, and especially from 3 to 10 minutes.

In another embodiment of the process, the processing composition containing the sample can be mixed with a PCR reaction mixture.

Said PCR reaction mixture preferably includes polymerase and primers and probes for detecting pathogenic microorganisms, especially pathogenic viruses, especially viruses that cause respiratory diseases.

In a specific embodiment, the process according to the invention includes the following steps:
a) providing a device according to the invention,
b) contacting a biological sample, preferably saliva and/or nasopharyngeal secretion, with the liquid processing composition for an in-situ (point of care; PoC) performance of a PCR test for determining coronaviruses, especially SARS CoV-2 viruses,
c) optionally incubating said liquid processing composition together with the biological sample,
d) mixing said liquid processing composition including the biological sample with a PCR reaction mixture including polymerase and primers and probes for detecting coronaviruses, especially SARS CoV-2 viruses.

Preferably, said PoC PCR method is a multiplex PCR method.

Said PCR reaction mixture includes 2 or more probes for the detection of SARS CoV-2 viruses, 2 or more primers, and polymerase, especially Taq polymerase, reverse transcriptase, and optionally a buffer.

The invention further relates to a kit comprising
a) a device according to the invention,
b) optionally a sample collecting device, and
c) optionally one or more separate containers with one or more PCR reaction mixtures.

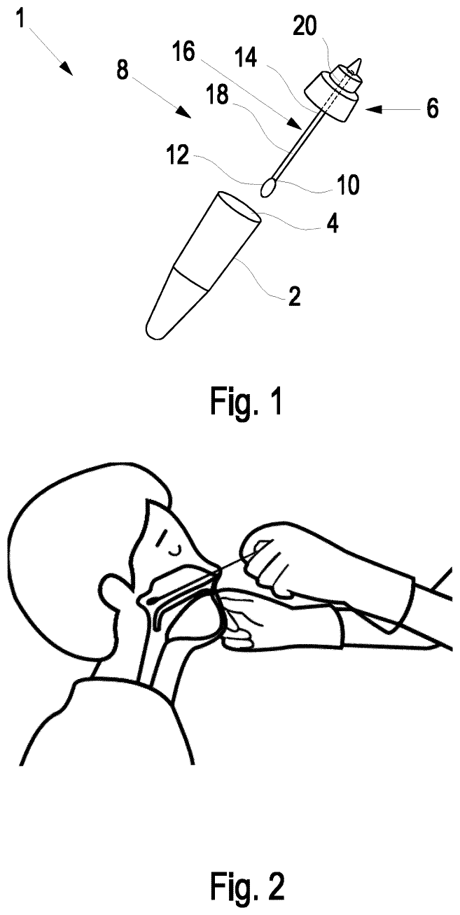

The embodiment represented in FIGS. 1 to 5 is explained in more detail below. The liquid processing composition is not shown in the Figures. It is provided in the receiving container.

Advantageously, the invention provides that the sample collecting means is connected with the closing means on a second end that is opposite to the first free end, so that the sample collecting means can be introduced into the receiving container by means of the closing means.

This embodiment of the present invention has the advantage that the collection of the samples is simplified. Said sample collecting means does not have to be placed elsewhere at first to open the closing means. An also, on the other hand, the closing means does not have to be placed anywhere for the sample collecting means to be introduced into the receiving container.

The sample collecting means may have at least one hollow rod-shaped element, at the free end of which said collecting element is provided, wherein said hollow rod-shaped element forms a canal inside.

Said collecting element may consist of a material that may be absorptive or adhesive with respect to liquids. That is to say, the collecting element can absorb the liquid, or the liquid adheres to the collecting element.

An absorbent material may be, for example, an element made of cotton. Alternatively, it may be any other element that can take up moisture.

The receiving container may be water-tight and airtight.

The closing means may have a passage opening.

The rod-shaped element can be connected with the closing means in such a way that the canal of the rod-shaped element leads to, or is provided in, the passage opening.

The passage opening of the closing element may be closed on the side opposite of the sample collecting element by means of a terminating element that is detachably attached to the closing element.

After separating off the terminating element, the passage opening is an opening towards the outside. With the terminating element separated off, processing composition liquid contained in the receiving container can flow because of capillary action and/or pressure and/or reduced pressure through the canal of the rod-shaped element, and exit to the outside through the passage opening.

The terminating element can be separable from the closing element by kinking. Alternatively, the terminating element can also be separable from the closing element by screwing.

Said hollow rod-shaped element may have at least one opening at the free end.

The end of the canal provided within said rod-shaped element may form the opening at the free end.

The opening provided at the free end may be provided in the wall of said rod-shaped element.

Further, a process for receiving samples, especially saliva samples, for detecting SARS-CoV-2 virus in the saliva may be provided, wherein said process comprises the following steps:
providing a receiving container with a first opening,
collecting a sample using at least one collecting element provided on a free end of a sample collecting means, wherein
the sample collecting means is connected with a closing means on a second end that is opposite to a first free end, and is introduced into the receiving container using said closing means, and said receiving container is closed by using the closing means.

The liquid can be introduced into the receiving container, and the collecting element of the sample collecting means can be immersed into the processing composition liquid when it is being introduced into the receiving container.

The liquid can be passed through a canal that is provided in a rod-shaped element of the sample collecting means to a passage opening of said closing means.

The liquid can be passed to said passage opening by capillary action and/or pressure and/or reduced pressure.

The passage opening may be closed with a terminating element, and said terminating element may be separated from said closing means.

The liquid that is passed through the canal provided in the rod-shaped element of the sample collecting means to the passage opening of the closing means can be discharged or poured out of the passage opening, after the terminating element has been separated from the closing means.

Commercially available devices, such as thermocyclers, are available for performing the POC PCR method. PCR is a widespread method in molecular biology, by means of which millions to billions of copies of a particular RNA/DNA sample can be prepared by thermocycles within extremely short periods of time.

Point of care (PoC) analytical devices have many advantages. They are mobile, lightweight, flexibly employable, and fast, above all. A result may be available as early as in 15 minutes. The name illustrates the most reasonable operation site of a PoC system: in immediate proximity to the patient and their treatment (point-of-care).

The mobile devices are quick testing systems for performing a qualitative detection of infectious diseases. The tests are performed on the basis of nucleic acid amplification technology (NAT). Therefore, the indication “PoC PCR device” is not explicitly found in some product descriptions. However, the PCR method is included in the term “nucleic acid amplification technology”. Most devices can detect not only Sars Cov-2, but also other diseases. For example, the ID Now™ device from Abbott can also detect influenza, the RS virus, or a Streptococcus A infection.

ID Now™ from Abbott is a molecular-diagnostic test system for the qualitative detection of infectious diseases, such as COVID-19 or influenza. Because of its isothermal nucleic acid amplification technology (NAT), the test device yields reliable results on a laboratory level.

Typical real-time qPCR systems for POC analysis may analyze several samples at a time, for example, 16 or 24 or 32. In addition, the devices preferably have channels for detection, for example, one or more channels for FAM/SYBR Green, VIC/JOE/HEX/TET, ABY/NED/TAMRA/Cy3, JUN, ROX/Texas RED, Cy5.

The reaction volume employed is typically from 5 μl to 50 μl.

The temperature rising rate with which the preferred thermocyclers can be operated is, for example, 5° C./s for heating; 5° C./s for cooling.

The PoC PCR analytical devices typically support DNA probes, preferably one or more selected from DNA binding dyes (e.g., SybrGreen), hydrolysis probes (e.g., TaqMan probe), and hybridization probes (e.g., FRET probes).

In a preferred embodiment of the present invention, said qPCR test device has a housing. Within the housing, a test module is provided for receiving at least one test sample, preferably 4 to 64, especially 16 to 24, test samples. A control module is connected with said test module, in which said control module is configured to control the test module, so that the test module performs qPCR tests for determining a test result. Preferably, the qPCR test device has a transmission module, which is connected with control module. Said transmission module is configured to transmit the test result and/or status information about the qPCR test device to at least one recipient. Thus, the qPCR test device is configured to transmit the test result to at least one recipient immediately and without manual transmission thereof according to a preferred embodiment of the present invention. Alternatively or in addition, status information about the qPCR test device can be transmitted to said at least one recipient by using the transmission module. Thus, the recipient can conclude that the qPCR test device is functional, whereby tampering with the qPCR test device is made difficult, for example. For example, said point-of-care qPCR test device may be placed in a pharmacy, a doctor's office, an ambulance, or in a mobile station, or the like. The sample collection then takes place at the same site. The sample is tested by using the qPCR test device. Then, the test result is transmitted automatically to at least one recipient. Said recipient may be, for example, a public/official agency, in particular, for registering notifiable diseases, or another registration agency/database for registering the test result. In particular, said transmission is effected in an anonymized way. Alternatively, said recipient may be the manufacturer of the qPCR test device, in which the test result and/or status information about the qPCR test device are transmitted. A manual and thus error-prone processing of the test results is no longer required. Also, automatized deposition into a central database or a central register is possible thereby, so that more test results can be processed in a shorter time. Further, the possibility of tampering with the test results can be reduced, because there is no intermediate step in which human workers are involved.

Preferably, the test module is configured to detect COVID, influenza and/or RSV (respiratory syncytial virus) infections. Thus, the qPCR test device can be configured to selectively detect one pathogen. Alternatively, the qPCR test device is configured to be able to detect different pathogens.

Preferably, the test module is configured to receive several test samples for testing. Thus, the test module can test several test samples simultaneously for a possible virus load. Thus, the processing speed is still increased, and test results are available earlier.

Preferably, one test result can be transmitted. Alternatively, a plurality of test results can be transmitted simultaneously. Also, the transmitted status information may contain a single status of the qPCR test device, which is then transmitted. Alternatively, the status information contains a plurality of different pieces of information about the status of the qPCR test device, which are preferably transmitted simultaneously.

Preferably, the transmission module is configured to set up a secure connection to said at least one recipient. For example, SSL is used between the qPCR test device and the recipient, in which a secure connection can be set up by exchanging certificates. By doing so, the possibilities for tampering are further reduced, and the safety of the data is increased.

Preferably, the transmission module is configured to transmit the test result to several recipients. Alternatively or in addition, the transmission module is configured to transmit status information to several recipients. Thus, it is possible that said qPCR test device transmits the test result, not just to one recipient, but the simultaneous provision of the test result and/or the status information to several recipients becomes possible. In particular, within the scope of a healthcare system that is interested in the monitoring of the spreading of possible diseases, it may be required to transmit test results to several recipients, especially in an anonymized way, in order to provide therefrom, for example, statistics, forecasts or information to the citizens about the spreading of the disease, and/or the burden to the healthcare system. A manual transfer of the test results within these information systems, which would lead to delays and errors, is no longer required.

Preferably, the status information includes one or more of a connection state of the qPCR test device, an error indicator, and a test performance indicator. Using the connection state, it can be established whether the qPCR test device is on-line and capable of transmitting test results by using the transmission module. Using the error indicator, error states of the qPCR test device can be indicated. In particular, if the status information is transmitted to the manufacturer of the qPCR test devices, they can already derive possible repair therefrom, and prepare it accordingly. At the same time, the error indicator ensures that only correct test results are forwarded. Thus, if the error indicator indicates an error that, in particular, could have an impact on the test result, then further tests by the qPCR test device are excluded, or their test results discarded. Using the test performance indicator, it is indicated whether the qPCR test device is currently performing a test. The activity of the qPCT test device can be further monitored thereby. In particular, a causal relationship between the performance of a test and the provision of test results can be created thereby, so that the provision of manipulated test results is further made difficult.

Preferably, the test results include one or more of a test indicator, a viral load, a personal ID, and a verification indicator. The test indicator yields an indication of a disease in the subject. In particular, the test indicator may have a binary design and thus represent the result of the evaluation of the qPCR test. Further, the test result may contain a viral load that was determined by the qPCR test. Further, the test result may contain a personal ID. In particular, said personal ID is an anonymized identification of the subject. Such personal ID can be made available, for example, as a bar code used to assign the samples. In particular, the personal ID does not include any person-related information (such as the name, address, passport number, or the like) and is set up, for example, by the test device. In particular, the personal ID includes a device ID of the qPCR test device. This ensures that the personal ID of the respective qPCR test device is unique, and a collision of personal IDs from different test devices is excluded. Alternatively, the personal ID is assigned centrally in order to ensure the uniqueness of the personal ID. Further, the test result may have a verification indicator. The verification indicator serves to indicate whether the sample is a sample from a human. This prevents the sample from being tampered with during the sample collection.

Preferably, the status information is transmitted on a regular basis to said at least one recipient, especially by means of an http inquiry or http request. This provides a simple transmission path for the status information to said at least one recipient. The status information can be transmitted to the at least one recipient, for example, once per hour, once per minute, or every 10 seconds. The present invention is not limited to any particular frequency of transmission of the status information, as long as this frequency is high enough to ensure a secure monitoring of the qPCR test device and its functionality.

Preferably, some piece of access information of a recipient is deposited in the transmission module or control module. Alternatively, the qPCR test device has a storage module, wherein said storage module is connected to said control module and/or the transmission module, and configured to store access data of a recipient. This additional information may include, for example, a password and/or a device ID. A transmission of test results and/or status information to the recipient is possible by using the access information. If the recipient is, for example, a public/official agency for registering the test results, or a data base, the access to this public agency or data base is protected. Only if the additional information is available, test results and/or status information can be transmitted to the central agency or recipient. If the access information does not match the access information deposited with the recipient for the corresponding qPCR test device, transmission of the test results and/or status information to this recipient is not possible.

Preferably, the transmission module is configured to transmit the access information for a second recipient to a first recipient. Thus, for example, access information can be transmitted automatically from the qPCR test device directly to the first recipient together with the test results and/or status information.

Then, further transmission to the second recipient takes place, in which the access information is used to forward the test results to the second recipient, or deposit them there as described above. The access information is transmitted from the qPCR test device to the first recipient in an encoded state, especially as a JASON Web Token, so that the first recipient has no knowledge about the access information. Decoding by the first recipient is not possible. Only by receiving the access information or the JASON Web Token, the second recipient can decode the encoded access information, which can then be used as access information for further processing/depositing the test result. Thus, the transmitted test results and/or status information can be received and further processed by the second recipient. This enables a flexible design of the flow of information from the qPCR test device to the several recipients. Thus, for example, a transmission of the test results and/or status information from the qPCR test device to the first recipient can take place at first, in particular, by means of a secured SSL connection, wherein said first recipient may be, for example, the manufacturer of the qPCR test device or a service provider. The latter then forwards the test results and/or status information to a second recipient. The second recipient may be, for example, a public/official agency, such as a health department, a pharmacy association, a medical association, or the like, who are supposed to further process the test results. The corresponding access data that include the access to the second recipient for depositing the test results are transmitted along by the qPCR test device, especially in an encoded form, for example, as a JASON Web Token. Thus, knowledge of the access data is just excluded for the manufacturer as the first recipient, since decoding of the access information by the first recipient does not take place, or is excluded. Nevertheless, forwarding to the respective second recipient is possible. If the test results are to be made available also to further or other second recipients, it is no longer required to reconfigure individual qPCR test devices. Rather, forwarding from the first recipient, such as the manufacturer or a service provider, to such new second recipients can then take place directly and centrally. Thus, the flow of information becomes flexible, and the adaptation for to whom the respective test results and/or status information are forwarded is easily possible.

Further, the present invention preferably relates to a system comprising a qPCR test device as described above, and at least one first recipient.

Preferably, said first recipient is configured to forward test results to a second recipient.

Preferably, said first recipient is a manufacturer of the qPCR test device, or a service provider. In particular, the second recipient may be a public/official agency, such as a health department, a medical association, a pharmacists association, or the like.

Further, the present invention relates to a process for transmitting test results of a point-of-care real-time quantitative PCR (qPCR) test device, comprising the steps of:
generating a test result and/or a piece of status information, and
transmitting the test result and/or piece of status information to at least one recipient.

Preferably, said qPCR test device is set up as described above.

Preferably, said transmission is effected by wire or wireless using known types of transmission, for example, through the internet using LAN, WLAN, a GSM standard, or another wireless transmission technology, such as Bluetooth, ZigBee, or the like. The transmission can be effected through several knots or access points. In particular, the present invention is not limited to the specific type of transmission of the test results and/or status information.

Preferably, the transmission is encoded or secured, especially by using SSL.

Preferably, the status information is transmitted to said at least one recipient on a regular basis.

Preferably, the test result is transmitted to several recipients. The transmission from the qPCT test device can be effected directly to several recipients. Alternatively, the forwarding of the test results is effected by one of the recipients.

Preferably, the transmission of the status information is effected to only one recipient.

Preferably, access information for access to one recipient is deposited on the qPCR testing device. Thus, the test results can be transmitted to the at least one recipient together with the access information. The access information is required to achieve deposition of the test results and/or status information with said at least one recipient.

Preferably, the access information is transmitted to a first recipient together with the test results and/or status information in an encoded form, especially by JASON Web Token. For example, the transmission can be effected with SSL securing. The test results are then transmitted to a second recipient by means of the access information, wherein the access information is required for deposition of the test results with said second recipient. Said first recipient may be, for example, the manufacturer of the qPCR test device or a service provider, wherein the second recipient may be, for example, a public/official agency, such as a health department, a pharmacy association, and/or a medical association. Thus, the deposition of the test results for further use by the second recipient can be performed only if the access information is available. The forwarding of the access information is done without the first recipient getting aware of this fact. Decoding the access information by the first recipient is not possible. Thus, the deposition of test results tampered with, for example, by the first recipient, is not possible, because they have no knowledge about the access information. The unintended reading of test results by the first recipient is not possible, either, since they have no access to the second recipient.

Preferably, said at least one recipient determines the consumption of test samples or further test material from the status information and/or planning information, which is transmitted by the qPCR test device and/or the operator of the qPCR test device to said at least one recipient. Said planning information may be, for example, information about the tests to be performed in the future. It can be forwarded by the operator of the qPCR device to the first recipient, for example, automatically. In particular, said recipient may be the manufacturer of the qPCR test device, or a service provider. If the number of available test samples or the availability of further test material falls below a predefined limit, a signal is triggered. Such signal may be an optical or acoustic signal, which is issued with said at least one recipient. Alternatively, said signal is a control signal, which triggers an automated provision of further test samples or further test material to the operator of the qPCR test device. In particular, from the status information and/or planning information, the presumable amount, required test samples and/or test material can be established, and thus, the number of test samples to be forwarded, or the extent of the test material to be forwarded has to be adapted.

In the following, Examples of the present invention are explained in more detail with reference to the drawings.

This is what the Figures show schematically:

FIG. 1: a device for receiving samples according to an embodiment of the present invention,

FIG. 2: the receiving of samples by means of an embodiment of the device according to the invention,

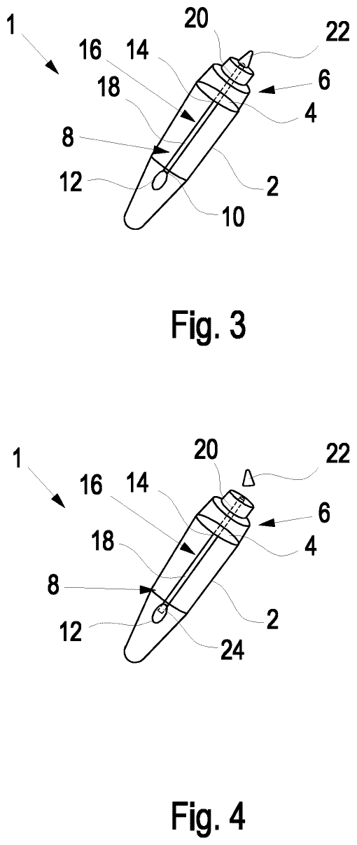

FIG. 3: an embodiment according to the invention of the device with a closed closing means,

FIG. 4: a device from FIG. 3 with detached terminating element,

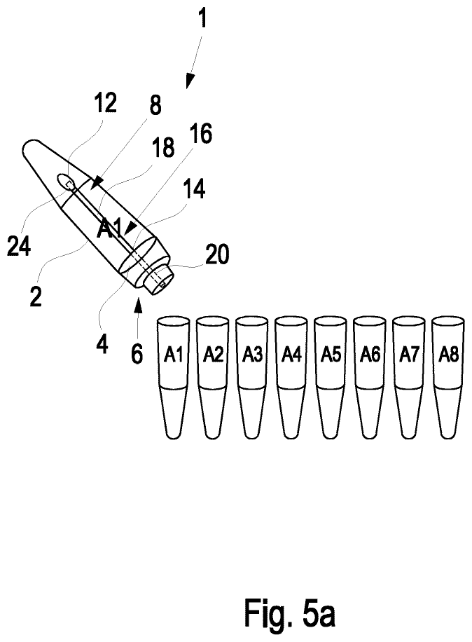

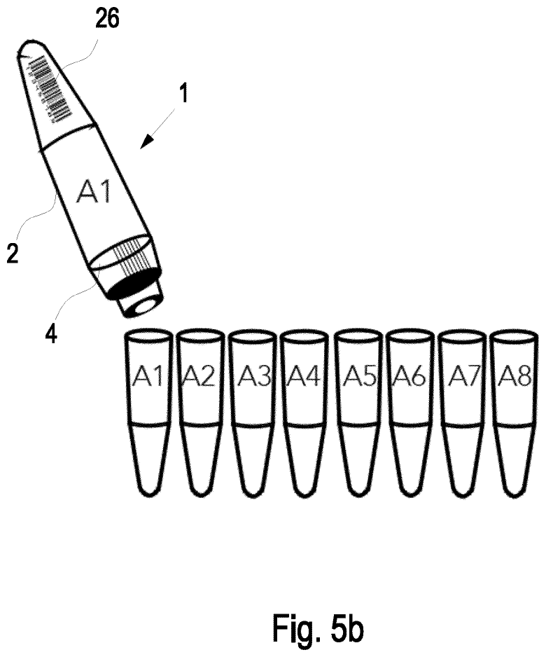

FIG. 5a: a device according to FIG. 4 in which the sample liquid to be analyzed is poured into a PCR vessel,

FIG. 5b: a device according to FIG. 9 in which the sample liquid to be analyzed is poured into a PCR vessel,

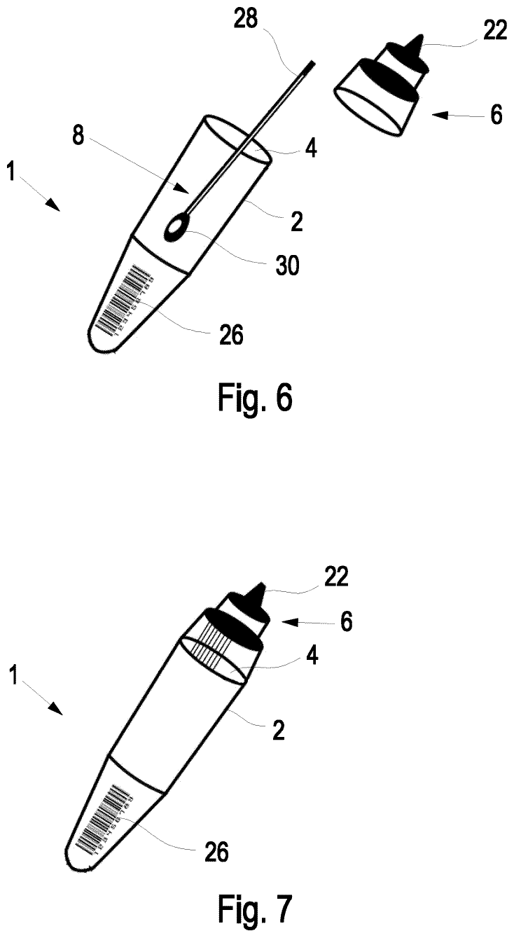

FIG. 6: a device according to FIG. 7 in opened state and with swab,

FIG. 7: an embodiment according to the invention of the device with a closed closing means,

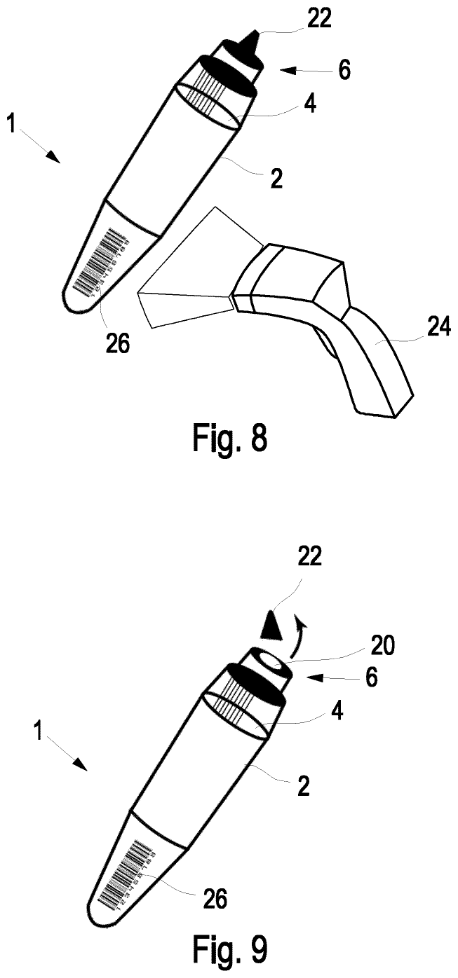

FIG. 8: scanning the identification code of an embodiment of a device according to the invention, and

FIG. 9: a device from FIG. 6 with detached terminating element,

FIG. 1 shows an embodiment of the device according to the invention for receiving samples, especially saliva samples or nasopharyngeal swab samples for performing a PCR analytical method for determining pathogenic microorganisms, in particular, pathogenic viruses, especially viruses causing respiratory diseases, especially coronaviruses, in particular, SARS CoV-2 viruses. The device has a receiving container 2, which has a first opening.

Further, a closing means 6 is provided that is adapted to the opening 4 of the receiving container 2, so that the opening 4 of the receiving container 2 can be closed by the closing means 6. Further, a sample collecting means 8 is shown that has at least one first collecting element 12 on a first free end 10. The sample can be collected with said collecting element 12. Said collecting element 12 may consist of a material that may be absorptive or adhesive with respect to liquids. That is to say, the collecting element can absorb the liquid, or the liquid adheres to the collecting element.

An absorbent material may be, for example, an element made of cotton. Preferably, said collecting element comprises cotton threads or Dacron fibers. In specific embodiments, the collecting element comprises microfibers. These improve collection and increase the collected volume of the sample material.

The sample collecting means 8 is connected with the closing means 6 on a second end 14 that is opposite to the first free end 10. The sample collecting means 8 can be introduced into the receiving container 2 by means of the closing means 6.

The sample collecting means 8 comprises at least one hollow rod-shaped element 16. The receiving element 12 is provided at the free end of the rod-shaped element 16. Said hollow rod-shaped element 16 forms a canal 18 inside. In this embodiment, the rod-shaped element 16 and thus the sample collecting means 8 are connected with the closing means 6.

Preferably, said rod-shaped element 16 includes a plastic material, preferably including polyethylene and/or polypropylene.

The closing means 6 has a passage opening 20. The sample collecting element and thus the rod-shaped element 16 are connected with the closing means 6 in such a way that the canal 18 leads to, or is provided in, the passage opening 20. Thus, said rod-shaped element 18 may be provided in the passage opening 20, and be connected with the closing means 6, or be connected with the closing means 6 at the end opposite to the sample collecting means 8, so that the canal 18 leads to the passage opening 20.

In FIG. 2, it is shown how the sample, especially a saliva sample, can be collected. For this, the sample collecting means 8 can be inserted through the nose or through the mouth, and the sample can be collected with said collecting element 12.

In FIG. 3, it is shown how the receiving container 2 is closed with the closing means 6. The closing means closes the opening 4. With a closing means, the receiving container 2 can be closed in a liquid-tight and preferably airtight way. The length of the sample collecting means 8 is selected in such a way that the sample collecting means 8 does not meet the bottom of the collecting container 2 when the closing means 6 is closed. Preferably, however, the sample collecting means 8 is so long that the liquid processing composition for the in situ (point of care; PoC) performance of a PCR test is in contact with the receiving element 12, or the sample collecting element is immersed in said liquid.

After a sample has been collected with the collecting element 12, the sample collecting means 8 can be introduced into the receiving container 2. By using the closing means 6, the opening 4 can be closed, and the entire device safely transported, without contaminants getting onto the sample.

According to the invention, the liquid processing composition for an in-situ (point of care; PoC) performance of a PCR test is provided within the receiving container 2, so that after the sample has been collected, the sample collecting means 8 is introduced into said receiving container 2, and the collecting element 12 is immersed directly into the liquid. The opening 4 can be closed by using the closing means 6.

FIG. 3 further shows a terminating element 22 that closes the passage opening 20 of the closing element 6 towards the outside. The terminating element 22 is detachably connected with the closing element 6. As long as the terminating element 22 is connected with the closing element 6, no liquid can get outside through the canal 18 and the passage opening 20.

FIG. 4 shows the device according to FIG. 3, but with the difference that the terminating element 22 has been separated from the closing element 6. Preferably, the terminating element 22 can be separated from the closing element 6 by kinking.

Said hollow rod-shaped element 16 may have at least one opening at the free end 10. This opening may form the opening of the canal provided within said rod-shaped element. Presently, this may be the opening 24, which is provided in said collecting element 12. The liquid that passes through the collecting element 12 can then be led to the passage opening 22 through the canal 18, and can leave the receiving container there when the terminating element 22 has been separated off. The liquid can be passed to said passage opening 22 by capillary action and/or pressure and/or reduced pressure, and can leave the receiving container 2 there when the terminating element 22 has been separated off.

This is shown, for example, in FIG. 5a. The processing composition for an in-situ (point of care; PoC) performance of a PCR test that has been contacted with the biological sample is poured out of the receiving container 2 there into a first PCR vessel A1. The latter can then be used further directly on the spot for a further PCR examination.

The opening in said rod-shaped element 16 may also be provided in the peripheral wall, and/or may also be provided in the zone adjacent to said closing means 6. Also, both at least one opening may be provided in this zone, and an additional opening may be provided in the zone of the collecting element 12.

FIG. 7 shows an embodiment of the device according to the invention for receiving samples, especially saliva samples or nasopharyngeal swab samples, for performing a PCR analytical method for determining pathogenic microorganisms, in particular, pathogenic viruses, especially viruses causing respiratory diseases, especially coronaviruses, in particular, SARS CoV-2 viruses. The device has a receiving container 2, which has a first opening.

Further, a closing means 6 is provided that is adapted to the opening 4 of the receiving container 2, so that the opening 4 of the receiving container 2 can be closed by the closing means 6.

The closing means 6 has a passage opening (not shown). Further shown is a terminating element 22 that closes the passage opening of the closing element 6 towards the outside. The terminating element 22 is detachably connected with the closing element 6. As long as the terminating element 22 is connected with the closing element 6, no liquid can get outside through the canal 18 and the passage opening 20.

Further, said receiving container may have an identification code 26.

FIG. 6 shows the embodiment according to FIG. 7, in which the closing element 6 has been separated from the receiving container 2. After a sample has been collected with the collecting element 30, the sample collecting means 8 can be introduced into the receiving container 2. The collected sample is contacted with the liquid processing composition for an in-situ (point of care; PoC) performance of a PCR test contained in the receiving container. The opening 4 can be closed by using the closing means 6, and the entire device can be transported safely without contaminants getting onto the sample.

According to the invention, the liquid processing composition for an in-situ (point of care; PoC) performance of a PCR test is provided within the receiving container 2, so that after the sample has been collected, the sample collecting means 8 is introduced into said receiving container 2, and the collecting element 30 is immersed directly into the liquid. The opening 4 can be closed by using the closing means 6. The sample collecting means 8 includes a rod-shaped element 28 and the collecting element 30.

FIG. 8 shows the embodiment according to FIG. 7 with an identification code 26, which is scanned and read by a scanner 24.

FIG. 9 shows the device according to FIG. 7, but with the difference that the terminating element 22 has been separated from the closing element 6. Preferably, the terminating element 22 can be separated from the closing element 6 by kinking.

FIG. 9 further shows a terminating element 22, which has been separated off and which previously closed the passage opening 20 of the closing element 6 towards the outside. The terminating element 22 is detachably connected with the closing element 6. As long as the terminating element 22 is connected with the closing element 6, no liquid can get outside through the passage opening 20.

The liquid processing composition for an in-situ (point of care; PoC) performance of a PCR test that has been contacted with the biological sample can be poured out of the receiving container 2 into a first PCR vessel A1 (FIG. 5b). The latter can then be used further directly on the spot for a further PCR examination.

Example: PoC PCR for SARS CoV-2 viruses

The device according to the invention for receiving biological samples as shown in FIGS. 7 and 9 contains an aqueous processing composition for the in-situ performance of a PCR test, comprising guanidinium thiocyanate, a surfactant, 2 different RNase inhibitors, an antibody that neutralizes SARS CoV-2 viruses, and biotin.

A nasopharyngeal secretion is collected from a patient with a swab, and contacted with the processing composition in the device according to the invention. The processing composition combined with the secretion is subsequently incubated at 20° C. for 5 minutes. One drop (30 μl) is charged through the passage opening in the closing means into a PCR sample vessel, which contains a PCR reaction mixture including polymerase and primers and probes for detecting SARS CoV-2 viruses. Subsequently, the PCR sample vessel is placed directly into a PoC PCR test device (thermocycler). The result is available after 10 to 30 minutes.

1. A device for receiving biological samples for performing a PCR analytical method for determining pathogenic microorganisms, comprising
a receiving container with a first opening,
a closing means that is adapted to the opening of the receiving container, so that the opening of the receiving container can be closed by the closing means, wherein said closing means has a passage opening, characterized in that said receiving container contains a liquid processing composition for the in-situ (point of care; PoC) performance of a PCR test.

2. The device according to claim 1, characterized in that the device further comprises
a sample collecting means that has at least one first collecting element on a first free end, with which the sample can be collected, and wherein the sample collecting means can be introduced into the receiving container.

3. The device according to claim 2, characterized in that the sample collecting means is connected with the closing means on a second end that is opposite to the first free end, so that the sample collecting means can be introduced into the receiving container by means of the closing means.

4. The device according to claim 1, characterized in that said passage opening of said closing means is closed by means of a terminating element, wherein said terminating element is detachably attached to said closing element.

5. The device according to claim 2, characterized in that the sample collecting means has at least one hollow rod-shaped element, at the free end of which said collecting element is provided, wherein said hollow rod-shaped element forms a canal inside.

6. The device according to claim 2, characterized in that said rod-shaped element is connected to said closing means in such a way that the canal of the rod-shaped element leads to, or is provided in, the passage opening.

7. The device according to claim 6, characterized in that the passage opening of the closing element is closed on the side opposite of the sample collecting element by means of a terminating element that is detachably attached to the closing element.

8. The device according to claim 4, characterized in that the terminating element is separable from the closing element by kinking.

9. The device according to claim 2, characterized in that said hollow rod-shaped element has at least one opening at the free end.

10. The device according to claim 9, characterized in that the end of the canal provided within said rod-shaped element forms the opening at the free end.

11. (canceled)

12. (canceled)

13. The device according to claim 1, characterized in that said viruses are coronaviruses.

14. The device according to claim 1, characterized in that one or more viruses are selected from the group consisting of SARS CoV-2 virus, influenza virus, and adenovirus.

15. The device according to claim 1, characterized in that said PCR test is a quantitative PCR (qPCR) method, or a quantitative real-time PCR (RT-qPCR).

16. The device according to claim 1, characterized in that said biological sample is saliva and/or nasopharyngeal secretion.

17. The device according to claim 1, characterized in that said liquid processing composition contains guanidinium thiocyanate, a buffer, biotin, or a combination thereof.

18. The device according to claim 1, characterized in that said liquid processing composition contains one or more surfactants or one or more RNase inhibitors.

19.-22. (canceled)

23. A process for performing an in-situ PCR (PoC PCR) method, comprising the following steps:
a) providing a device according to claim 1,
b) contacting a biological sample with the liquid processing composition for an in-situ (point of care; PoC) performance of a PCR test, and
c) using said liquid processing composition including the biological sample, in a PoC PCR method.

24. The process according to claim 23, characterized in that said liquid processing composition is incubated together with the sample at 15 to 30° C. over a period of from 1 to 30 minutes.

25. The process according to claim 23, characterized in that the processing composition containing the sample is mixed with a PCR reaction mixture comprising polymerase, primers, and probes for detecting pathogenic microorganisms.

26. (canceled)

27. A process for performing an in-situ PCR (PoC PCR) method, comprising the following steps:
a) providing a device according to claim 1,
b) contacting a biological sample with the liquid processing composition for an in-situ (point of care; PoC) performance of a PCR test for determining coronaviruses,
c) optionally incubating said liquid processing composition together with the biological sample, and
d) mixing said liquid processing composition including the biological sample with a PCR reaction mixture including polymerase and primers and probes for detecting coronaviruses.

28.-30. (canceled)
